(19) United States
(12) Patent Application Publication
(10) Pub. No.: US 2002/0006401 A1
(43) Pub. Date: Jan. 17, 2002

(54) Modulation of vascular healing by inhibition of leukocyte adhesion and function

(75) Inventors: Campbell Rogers, Westwood, MA (US); Elazer R. Edelman, Brookline, MA (US); Daniel I. Simon, Waban, MA (US)

Correspondence Address: ARNALL GOLDEN & GREGORY, LLP, 2800 ONE ATLANTIC CENTER, 1201 WEST PEACHTREE STREET, ATLANTA, GA, 30309-3450, US

(73) Assignee: Massachusetts Institute of Technology

(21) Appl. No.: 09/776,533

(22) Filed: Feb. 7, 2001

Related U.S. Application Data
(63) Continuation of application No. 08/823,999, filed on Mar. 25, 1997.

(51) Int. Cl. A61K 39/395

(52) U.S. Cl. 424/130.1

(57) ABSTRACT

Compounds that specifically inhibit or reduce leukocyte adhesion or function are useful to enhance vascular healing and lessen restenosis of blood vessels after revascularization, via angioplasty or bypass surgery, of diseased coronary, peripheral and cerebral arteries, and lessen stenosis or restenosis of surgically-placed bypass grafts and transplanted organs. Examples of these compounds are those which block cell surface integrins or their ligands, for example, the leukocyte integrin Mac-1 (CD11b/CD18, αMβ2). As demonstrated by the examples, both superficial and deep injury was significantly reduced with treatment using an antibody to Mac-1 compared to both saline controls and IgG controls. After balloon angioplasty (superficial injury) neointimal area was reduced nearly 70%. The ratio of intimal:medial area, which is customarily used in balloon-injured experimental arteries to normalize for small normal variations in arterial size from one animal to another, was reduced over 75%. After endovascular stent implantation (deep injury) neointimal area was reduced nearly 40%. Extrapolated to humans, this reduction in the intimal thickening would reduce restenosis from occurring in approximately 30% of patients to less than 10% of patients.

The United States government has rights in this invention by virtue of National Institutes of Health grants GM/HL 49039 to Elazer R. Edelman, HL03104 to Campbell Rogers and HL02768 to Daniel Simon.

### BACKGROUND OF THE INVENTION

The present invention is generally in the area of methods and compositions to reduce restenosis after revascularization of diseased coronary, peripheral, and cerebral arteries, and stenosis or restenosis of surgically-placed bypass grafts or transplanted organs.

Angioplasty, surgery and other vascular interventions are complicated by an accelerated arteriopathy characterized by rapid growth of cells into the lumen within a short period of time. This growth is often severe enough to jeopardize the blood flow to distal organs.

Vascular bypass surgery has been widely used to treat stenotic and occluded blood vessels, as when plaques develop on the surface of blood vessels in atherosclerosis. In bypass surgery, one or more healthy blood vessels are grafted into the stenotic/occluded vessels beyond the site of stenosis or occlusion to shunt blood around the stenotic or occluded vessel to re-establish a sufficient blood supply to the tissue whose blood supply is endangered by the stenosis or occlusion. This surgery often successfully revascularizes the endangered tissue.

In recent years, angioplasty has been developed as an alternative treatment to bypass surgery, especially in patients who have been diagnosed early in the development of stenosis or occlusion of blood vessels due to the abnormal laying down of plaque on the luminal wall of a blood vessel. Angioplasty typically involves guiding a catheter which is usually fitted with a balloon or expandable metal mesh up through an artery to the region of stenosis or occlusion and the brief inflation, one or more times, of the balloon or wire mesh to push the obstructing intravascular material or plaque up against the endothelial wall of the vessel, thereby compressing and/or breaking apart the plaque and reestablishing blood flow. However, angioplasty treatment can injure the vessel, especially when the balloon is overinflated or the mesh overextended, causing a variety of undesirable results, such as denudation (removal) of the endothelial cell layer in the region of the angioplasty, dissection of part of the inner vessel wall from the remainder of the vessel with accompanying occlusion of the vessel, or rupture of the tunica intima layer of the vessel.

Injury of arteries in animals induces a process of vascular repair which eventually causes the artery to become narrowed. A thick new layer, or neointima, of smooth muscle cells and inflammatory cells grows within the blood vessel, encroaching on the lumen. This process in animals represents the process which occurs clinically after angioplasty, endovascular stent implantation, organ transplantation, or bypass surgery, which greatly limits the long term successes of these techniques for treating obstructive arterial disease. Animal models of arterial injury and neointimal hyperplasia have been used to study the cellular events which lead to restenosis in humans, to devise treatment strategies to suppress tissue growth in an attempt to reduce restenosis and enhance long term clinical results.

Attempts to limit stenosis or restenosis of blood vessels following revascularization have included administration of pharmacologic agents and technical approaches. No pharmacologic agent has yet been shown to reduce restenosis in humans. One technical approach, endovascular stent placement, has been shown to partially reduce restenosis in humans after coronary arterial intervention, as reported by Serruys, et al. N. E. J. Med. 1994; 331:489-495 and Fischman, et al. N. E. J. Med. 1994; 331:496-501. Nevertheless, stents themselves remain susceptible to significant restenosis in 20-30% of cases.

Increased knowledge of the mechanisms underlying vascular repair has led to innovative proposals for agents to limit accelerated arteriopathies. Circulatory leukocytes, including monocytes, are known to be among the very first cells recruited to blood vessels as atherosclerosis begins. Once within diseased arterial walls, these cells may engulf cholesterol and other lipids, and may also produce substances which attract other cells, cause other cells to proliferate, or degrade matrix components. Each of these secondary effects may in turn promote greater intimal thickening and more severe narrowing or occlusion of the arterial lumen.

A similar role for leukocytes in restenosis after revascularization has not been proven. Although leukocyte activation has been connected to restenosis in humans (Pietersma, et al. Circulation 1995; 91:1320-1325; Mickelson, et al., 1996 JACC 28(2):345-353; Inoue, et al., 1996 JACC 28(5):1127-1133) broad inhibition of inflammation, for example with glucocorticoids, after revascularization has not reduced restenosis in humans (Pepine et al., Circulation 1990; 81:1753-1761). This observation is reminiscent of studies using both broadly active and very specifically targeted treatments for preventing restenosis. Broad treatments, for example with heparin, have been limited by systemic toxicities and dosing limitations. Specific treatments, for example with molecular strategies, have failed to inhibit all of the redundant cellular and molecular pathways which activate and potentiate the vascular repair process.

Accordingly, there is a need for compositions and methods of promoting healing of vascular tissue and controlling vascular muscle cell proliferation (hyperplasia) to prevent restenosis of blood vessels after angioplasty, vascular bypass, organ transplantation, or vascular disease, with minimal risk of rapid reocclusion.

It is therefore an object of the present invention to provide a method and compositions to reduce restenosis after revascularization of diseased coronary, peripheral, and cerebral arteries and stenosis or restenosis of surgically-placed bypass grafts or transplanted organs.

### SUMMARY OF THE INVENTION

Compositions and methods for reducing stenosis or restenosis after revascularization of diseased coronary, peripheral, and cerebral arteries and stenosis or restenosis of surgically-placed bypass grafts or transplanted tissues are described which involve administration of a composition specifically inhibiting integrin-mediated leukocyte adhesion or function, prior to, at the time of and/or subsequent to vascular intervention.

Leukocyte adhesion or function can be inhibited or reduced by blocking cell surface integrins, most preferably the leukocyte integrins Mac-1 (CD11b/CD18, αMβ2), LFA-1 (CD11a/CD18, αLβ2), p150,95 (CD11c/CD18, αXβ2) and, potentially, CD11d/CD18, or their ligands. Ligands for Mac-1 include, among others, ICAM-1, fibrin(ogen), C3bi, and factor X. Ligands for LFA-1 include ICAM-1, ICAM-2, and ICAM-3. Ligands for p150,95 include fibrin(ogen) and C3bi. Mac-1 also regulates Urokinase Plasminogen Activator Receptor (uPAR) mediated adhesion to vitronectin or serum.

Exemplary compounds for inhibiting or reducing leukocyte adhesion or function include antibodies and antibody fragments that are immunoreactive with these integrins or their ligands and which inhibit or reduce the binding of integrins or their ligands to vascular cells; molecules which inhibit or reduce the expression of the integrins or their ligands, including nucleic acid regulators such as antisense oligonucleotides, ribozymes and external guide sequences for RNAase P, molecules involved in triplex formation, aptamers, peptides and peptidomimetics derived from the integrins or their ligands which block the interaction of the integrins or their ligands with vascular cells such as peptides and peptidomimetics that block the leukocyte integrin Mac-1. The compounds can be administered systemically or administered directly to the site of vascular injury, most preferably prior to and after injury.

Example 1 demonstrates that an antibody to Mac-1 (M 1/70) binds to rabbit peripheral blood mononuclear cells and thereby inhibits ligand binding to Mac-1. Serum obtained from rabbits after intravenous bolus administration of M1/70 (1 mg/kg) is capable of inhibiting Mac-1 function. Example 2 demonstrates the neointimal hyperplasia after both superficial and deep injury was significantly reduced with M1/70 treatment, as compared to both saline controls and IgG controls. After balloon angioplasty (superficial injury), neointimal area was reduced nearly 70% relative to controls. The ratio of intimal:medial area, which is customarily used in balloon-injured experimental arteries to normalize for small normal variations in arterial size from one animal to another, was reduced over 75% relative to controls. After endovascular stent implantation (deep injury), neointimal area was reduced nearly 40% relative to controls.

### BRIEF DESCRIPTION OF THE DRAWINGS

FIG. 1 is a graph of Mac-1-dependent ligand binding in rabbit monocytes showing % fibrinogen binding in the presence of M1/70 (1-5 μg/ml) or control mAb M5/14 (5 μg/ml).

FIG. 2 is a graph of Mac-1-dependent fibrinogen binding in cultured macrophages showing the degree of inhibition as a function of M 1/70 concentration. Serum level of M1/70 after intravenous bolus administration in two separate rabbits is estimated by extrapolating from the degree of inhibition of fibrinogen binding to Mac-1.

FIG. 3 is a graph of intimal area (mm²) versus monocytes (cells per section).

FIG. 4A is a graph of the response to balloon-induced superficial injury showing the neointimal area (mm²) after 14 days of treatment with M1/70, saline and IgG.

FIG. 4B is a graph of the response to balloon-induced superficial injury showing the ratio of intimal-medial area (mm²) after 14 days of treatment with M1/70, saline and IgG.

FIG. 4C is a graph of the response to stent-induced deep injury showing the neointimal area (mm²) after 14 days of treatment with M1/70, saline and IgG.

### DETAILED DESCRIPTION OF THE INVENTION

The compositions described herein are used to inhibit undesired response to vascular injury that includes hyperplasia of vascular smooth muscle cells which occurs in response to injury of blood vessels, for example, as a result of angioplasty, atherectomy, endovascular stenting coronary or peripheral arterial bypass used to open a stenotic or occluded vessel or transplantation of cells, tissue or organs. Vascular smooth muscle cell hyperplasia triggered by the injured vessel can result in stenosis or restenosis of the blood vessel. These compositions and methods are based on the discovery that inhibition of integrin-mediated leukocyte adhesion and/or function, especially adhesion and function of monocytes and granulocytes, can significantly reduce restenosis.

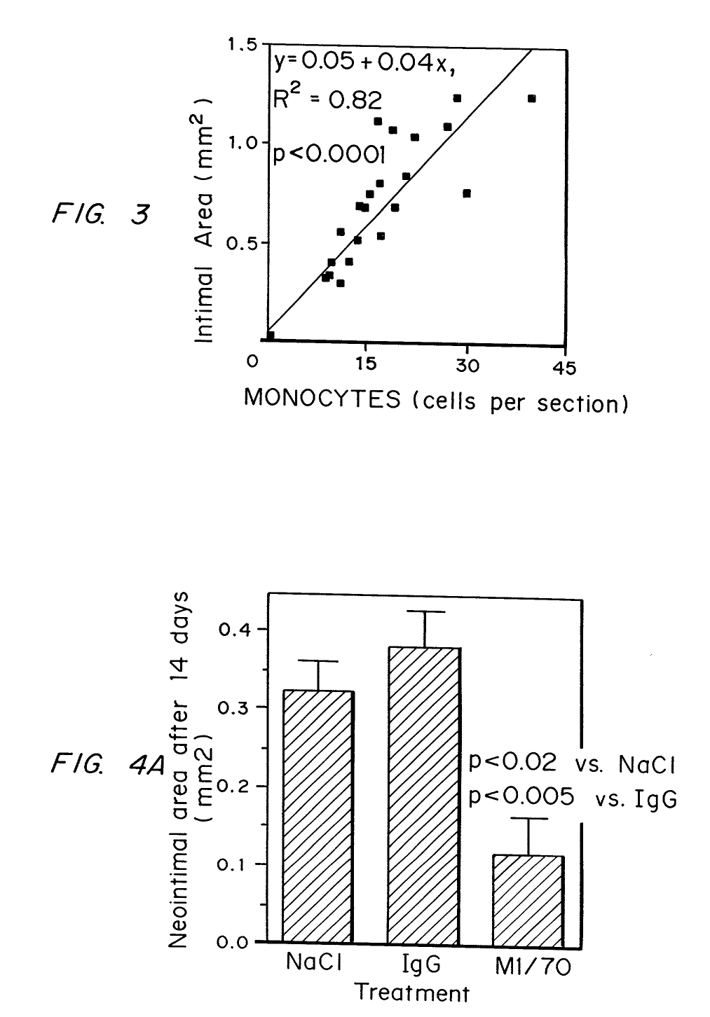

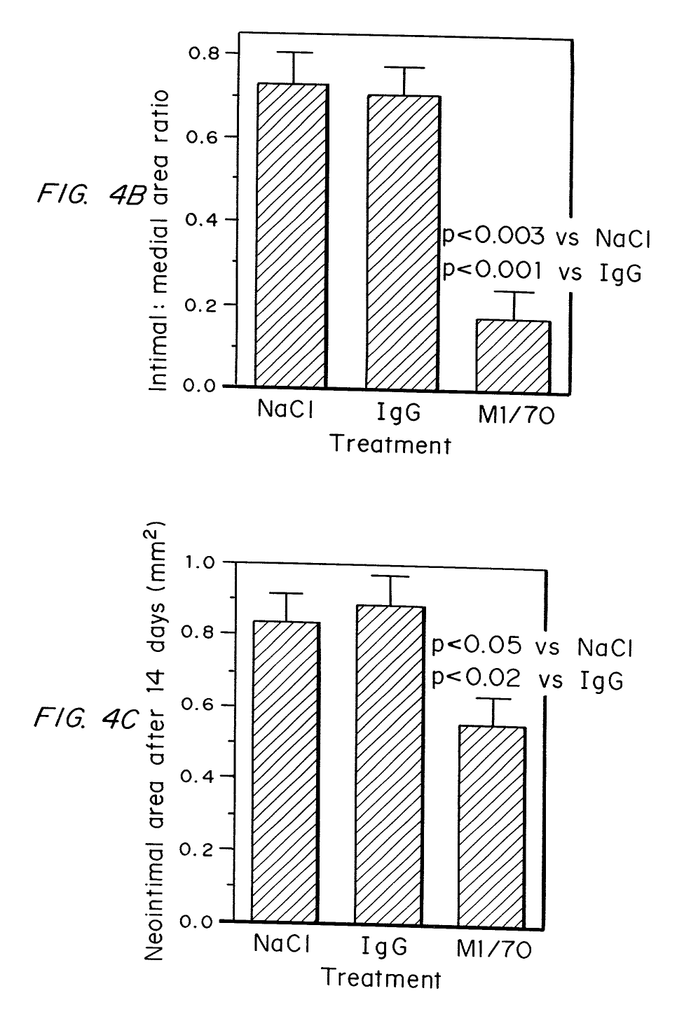

Restenosis is an extremely complex phenomenon, involving numerous complex interactions. Many “single target” therapies have been tried as a means to reduce the occurrence or severity of restenosis, unsuccessfully. The extent of neointimal hyperplasia and cellular proliferation in animal models of vascular injury and repair is associated with the number of adherent and infiltrating monocytes, as reported by Rogers et al., Circulation 1995; 91:2995-3001, and as demonstrated by FIG. 3. FIG. 3 shows that as the intimal area increases, the number of monocytes also increases. Pharmacological inhibition of neointimal hyperplasia and monocyte adhesion/infiltration with heparin are commensurate with one another, as reported by Rogers et al., Art. Thromb. Vasc. Bio. 1996, 16:1312-1318.

As described herein, it has now been demonstrated that this process is involved in restenosis and that inhibition of integrin-mediated leukocyte adherence or function can be used to decrease the amount of neointima formed following vascular injury. These results are particularly striking in view of the complexity of the problem and the lack of success previously achieved using compounds blocking specific sites.

I. Composition

Compositions useful as described herein include one or more compounds that inhibit or reduce leukocyte adhesion or function by interference with integrin-mediated binding. Leukocyte adhesion and function can be inhibited or reduced by blocking cell surface integrins, such as the leukocyte integrin Mac-1 (CD11b/CD18, αMβ2), described by Diamond et al., J. Cell Bio. 1993; 120:1031-1043, LFA-1 (CD11a/CD18, αLβ2), p150,95 (CD11c/CD18, αXβ2) and potentially CD11d/CD18, or their ligands. Ligands for Mac-1 include, among others, ICAM-1, fibrin(ogen) C3bi, and factor X. Ligands for LFA-1 include ICAM-1, ICAM-2, and ICAM-3. Ligands for p150,95 include fibrin(ogen) and C36. Mac-1 also regulates Urokinase Plasminogen Activator Receptor (uPAR) mediated adhesion to vitronectin or serum.

Suitable compounds include antibodies and antibody fragments that are immunoreactive with integrins or their ligands and which inhibit or reduce the binding of integrins or their ligands to vascular cells; molecules which inhibit or reduce the expression of integrins or their ligands, including nucleic acid regulators such as antisense oligonucleotides, ribozymes and external guide sequences for RNAase P, molecules involved in triplex formation, aptamers, and peptides and peptidomimetics derived from the integrins or their ligands which block the interaction of the integrins or their ligands with vascular cells.

a. Compounds Blocking Leukocyte Adherence and/or Function

Integrins Involved in Leukocyte Adherence and Function

The specific adhesion of cells to other cells or to extracellular matrices is a basic component of cell migration and recognition. Many different genes have evolved that encode proteins with specific adhesive functions. These genes display homologies indicative of a common ancestral gene. The integrin superfamily consists of about 30 structurally homologous proteins that promote cell-cell or cell-matrix interactions. All integrins are heterodimeric cell surface proteins composed of two non-covalently linked polypeptide chains, α and β. The α chain varies from 120 to 200 kD and the β chain varies from 90 to 110 kD. The N-terminus of each chain forms a globular head that contributes to the interchain linking and to ligand binding. The α subunits contain divalent cation-binding domains which are essential for integrin receptor functions. Stalks extend from the globular heads to the plasma membrane, followed by transmembrane segments and cytoplasmic tails, which are usually less than 50 amino acid residues long. The cytoplasmic domains of the integrins interact with cytoskeletal components such as vinculis, talin, actin, alpha-actinic, and tropomyosin, and it is hypothesized that the integrins coordinate the binding of cells to extracellular proteins with cytoskeleton-dependent motility, shape change, and phagocytic responses.

Mac-1, also known as CD11b/CD18, CR3, and αm/β2, is a leukocyte adhesion molecule found on monocytes, neutrophils, and natural killer lymphocytes. It binds heterogeneous ligands including, among others, fibrin(ogen), factor X, intercellular adhesion molecule-1 (ICAM-1), C3bi, and high-molecular-weight-kininogen.

Other integrins involved in leukocyte adhesions include LFA-1 (CD11a/CD18, αLβ2), p150,95 (CD11c/CD18, αXβ2), and potentially CD11d/CD18, or their ligands. Ligands for Mac-1 include ICAM-1, fibrin(ogen), C3bi, and factor X. Ligands for LFA-1 include ICAM-1, ICAM-2, and ICAM-3. Ligands for p150,95 include fibrin(ogen) and C3bi. Mac-1 also regulates Urokinase Plasminogen Activator Receptor (uPAR) mediated adhesion to vitronectin or serum.

Antibodies and Antibody Fragments

Antibodies, immunoreactive antibody fragments (including single chain recombinant antibodies) and humanized or chimeric antibodies are available, or readily constructed using known methodology and commercially available reagents, which react with either the integrins or their ligands are particularly useful. Other compounds which bind to the integrins or their ligands or which competitively inhibit binding of the integrins, such as peptide fragments derived from either the integrins or their ligands can also be used. Compounds which act at a more basic level by inhibiting expression of the integrins in their ligands can be designed based on the published DNA sequences encoding the integrins or their ligands. These compounds can be stabilized using routine technology to increase in vivo life.

A preferred integrin to inhibit is Mac-1. Mac-1 antibodies are described in the literature, for example, by Diamond, et al., J. Cell Bio. 1993; 120:1031-1043; Ault and Springer, J. Immunol. 1981; 120:359, 364; Anderson, et al., J. Immunol. 1986, 137:15-27; and Altieri, et al., J. Cell Biol. 1988, 107:1893-1900. An exemplary antibody is M1/70, a rat-derived IgG2b monoclonal antibody (mAb) directed to the αM-subunit (CD11b) of mouse Mac-1 that has broad species reactivity and blocks ligand binding to Mac-1. This antibody was obtained from a hybridoma derived by fusion between immune rat spleen cells and the mouse NSI myeloma line. This antibody immunoprecipitates two polypeptides from leukocytes (190 kD and 105 kD). The antigenic determinant defined by M1/70 is expressed on neutrophils, macrophages, monocytes, and NK cells. M1/70 is cross-reactive with human Mac-1, and blocks the binding of multiple ligands to Mac-1, thereby influencing adhesion, coagulation, complement binding and phagocytosis and homotypic leukocyte aggregation.

Antibodies to the integrin proteins which are useful for inhibition or reduction of binding are available or generated by standard techniques, using human or animal integrin proteins. Since the proteins exhibit high evolutionary conservation, it may be advantageous to generate antibodies to a protein of a different species of origin than the species in which the antibodies are to be tested or utilized, looking for those antibodies which are immunoreactive with the most evolutionarily conserved regions. Antibodies are typically generated by immunization of an animal using an adjuvant such as Freund's adjuvant in combination with an immunogenic amount of the protein administered over a period of weeks in two to three week intervals, then isolated from the serum, or used to make hybridomas which express the antibodies in culture. Methods for “humanizing” antibodies, or generating less immunogenic fragments of non-human antibodies, are well known.

For example, the CDR grafting method described by Daugherty, et al., 1991 Nucl. Acids Res., 19:2471-2476 may be used. Briefly, the variable region DNA of a selected animal recombinant anti-idiotypic ScFv is sequenced by the method of Clackson, T., et al., 1991 Nature, 352:624-688. Using this sequence, animal CDRs are distinguished from animal framework regions (FRs) based on locations of the CDRs in known sequences of animal variable genes. Kabat, H. A., et al., Sequences of Proteins of Immunological Interest, 4th Ed. (U.S. Dept. Health and Human Services, Bethesda, Md., 1987). Once the animal CDRs and FR are identified, the CDRs are grafted onto human heavy chain variable region framework by the use of synthetic oligonucleotides and polymerase chain reaction (PCR) recombination. Codons for the animal heavy chain CDRs, as well as the available human heavy chain variable region framework, are built in four (each 100 bases long) oligonucleotides. Using PCR, a grafted DNA sequence of 400 bases is formed that encodes for the recombinant animal CDR/human heavy chain FR protection.

These antibodies can be further modified by the use of Pharmacia's (Pharmacia LKB Biotechnology, Sweden) “Recombinant Phage Antibody System” (RPAS), which generates a single-chain Fv fragment (ScFv) which incorporates the complete antigen-binding domain of the antibody. In the RPAS, antibody variable heavy and light chain genes are separately amplified from the hybridoma mRNA and cloned into an expression vector. The heavy and light chain domains are co-expressed on the same polypeptide chain after joining with a short linker DNA which codes for a flexible peptide. This assembly generates a single-chain Fv fragment (ScFv) which incorporates the complete antigen-binding domain of the antibody.

Peptide and Peptidometic Compound

Compounds which are effective for blocking binding of the integrins or their ligands can also consist of fragments of the integrins or their ligands, expressed recombinantly and cleaved by enzymatic digest or expressed from a sequence encoding a peptide of less than the full length integrins or their ligands. These will typically be soluble proteins, i.e., not including the transmembrane and cytoplasmic regions, although smaller portions determined in the assays described above to inhibit or compete for binding to the integrins or their ligands can also be utilized. It is a routine matter to make appropriate fragments, test for binding, and then utilize. The preferred fragments are of human origin, in order to minimize potential immunological response. The peptides can be as short as five to eight amino acids in length and are easily prepared by standard techniques. They can also be modified to increase in vivo half-life, by chemical modification of the amino acids or by attachment to a carrier molecule or inert substrate. Based on studies with other peptide fragments blocking binding, the IC₅₀, the dose of peptide required to inhibit binding by 50%, ranges from about 50 μM to about 300 μM, depending on the peptides. These ranges are well within the effective concentrations for the in vivo administration of peptides, based on comparison with the RGD-containing peptides, described, for example, in U.S. Pat. No. 4,792,525 to Ruoslaghti, et al., used in vivo to alter cell attachment and phagocytosis.

The peptides can also be conjugated to a carrier protein such as keyhole limpet hemocyanin by its N-terminal cysteine by standard procedures such as the commercial Imject kit from Pierce Chemicals or expressed as a fusion protein, which may have increased efficacy.

As noted above, the peptides can be prepared by proteolytic cleavage of the integrins or their ligands, or, preferably, by synthetic means. These methods are known to those skilled in the art. An example is the solid phase synthesis described by J. Merrifield, 1964 J. Am. Chem. Soc. 85, 2149, used in U.S. Pat. No. 4,792,525, and described in U.S. Pat. No. 4,244,946, wherein a protected alpha-amino acid is coupled to a suitable resin, to initiate synthesis of a peptide starting from the C-terminus of the peptide. Other methods of synthesis are described in U.S. Pat. Nos. 4,305,872 and 4,316,891. These methods can be used to synthesize peptides having identical sequence to the proteins described herein, or substitutions or additions of amino acids, which can be screened for activity as described above.

The peptide can also be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Peptides containing cyclopropyl amino acids, or amino acids derivatized in a similar fashion, can also be used. These peptides retain their original activity but have increased half-lives in vivo. Methods known for modifying amino acids, and their use, are known to those skilled in the art, for example, as described in U.S. Pat. No. 4,629,784 to Stammer.

An example of a fibrinogen fragment shown to modify binding or fibrinogen to Mac-1 is described by Altieri, et al., J. Biol. Chem. 1993; 268:1847-1853 (WPVFQKLRLDSV). The binding regions of most of the integrins and their ligands have been identified or can readily be determined using peptide fragments and competitive binding assays.

Screening for drugs modifying or altering the extent of integrin function or expression

The integrin proteins are useful as targets for compounds which turn on, or off, or otherwise regulate binding to these integrins. A compound can be tested for an inhibitory effect on binding using routine methodology. The in vitro studies of compounds which appear to inhibit or reduce binding selectively to the integrins are then confirmed by animal testing. Since the molecules are so highly evolutionarily conserved, it is possible to conduct studies in laboratory animals to predict the effects in humans.

Assays for testing compounds for useful activity can be based solely on the interaction of the compound with the integrin protein, preferably expressed on the surface of cells in animals such as those described in the examples, although proteins in solution or immobilized on inert substrates can also be used.

Alternatively, the assays can be based on the interaction of the compound with the gene sequence encoding the integrin protein, preferably the regulatory sequences directing expression of the integrin protein. For example, antisense oligonucleotides which bind to the regulatory sequences, and/or to the protein encoding sequences, can be synthesized using standard oligonucleotide synthetic chemistry. The antisense oligonucleotides can be stabilized for pharmaceutical use using standard methodology (encapsulation in a liposome or microsphere; introduction of modified nucleotides that are resistant to degradation or groups which increase resistance to endonucleases, such as phosphorothioates and methylation), then screened initially for alteration of integrin activity in transfected or naturally occurring cells which express the integrin, then in vivo in laboratory animals. Typically, the antisense oligonucleotides would inhibit expression. However, sequences which block those sequences which “turn off” synthesis can also be targeted, resulting in increased expression.

Random generation of integrin or integrin encoding sequence binding molecules.

Molecules with a given function, catalytic or ligand-binding, can be selected for from a complex mixture of random molecules in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. For example, by repeated cycles of affinity chromatography and PCR amplification of the molecules bound to the ligand on the column, Ellington and Szostak (1990) estimated that 1 in 10¹⁰ RNA molecules folded in such a way as to bind a given ligand. DNA molecules with such ligand-binding behavior have been isolated (Ellington and Szostak, 1992; Bock et al, 1992).

Computer assisted drug design

Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to a target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Ann. Rev. Pharmacol. Toxicol. 29, 111-122; Perry and

Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model integrin for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Generation of nucleic acid regulators

Nucleic acid molecules containing the 5′ regulatory sequences of the integrin genes can be used to regulate or inhibit gene expression in vivo. Vectors, including both plasmid and eukaryotic viral vectors, may be used to express a particular recombinant 5′ flanking region-gene construct in cells depending on the preference and judgment of the skilled practitioner (see, e.g., Sambrook et al., Chapter 16). Furthermore, a number of viral and nonviral vectors are being developed that enable the introduction of nucleic acid sequences in vivo (see, e.g., Mulligan, 1993 Science, 260, 926-932; U.S. Pat. Nos. 4,980,286; 4,868,116. Delivery systems in which nucleic acid is encapsulated in cationic liposomes which can be injected intravenously into a mammal are commercially available. This system has been used to introduce DNA into the cells of multiple tissues of adult mice, including endothelium and bone marrow (see, e.g., Zhu et al., 1993 Science 261, 209-211.

The 5′ flanking sequences of the integrin gene can also be used to inhibit the expression of the integrin. For example, an antisense RNA of all or a portion of the 5′ flanking region of the integrin gene can be used to inhibit expression of the integrin in vivo. Expression vectors (e.g., retroviral expression vectors) are already available in the art which can be used to generate an antisense RNA of a selected DNA sequence which is expressed in a cell (see, e.g., U.S. Pat. Nos. 4,868,116; 4,980,286). Accordingly, DNA containing all or a portion of the sequence of the 5′ flanking region of the integrin gene can be inserted into an appropriate expression vector so that upon passage into the cell, the transcription of the inserted DNA yields an antisense RNA that is complementary to the mRNA transcript of the integrin protein gene normally found in the cell. This antisense RNA transcript of the inserted DNA can then base-pair with the normal mRNA transcript found in the cell and thereby prevent the mRNA from being translated. It is of course necessary to select sequences of the 5′ flanking region that are downstream from the transcriptional start sites for the integrin protein gene to ensure that the antisense RNA contains complementary sequences present on the mRNA.

Antisense RNA can also be generated in vitro, and then inserted into cells. Oligonucleotides can be synthesized on an automated synthesizer (e.g., Model 8700 automated synthesizer of Milligen-Biosearch, Burlington, Mass. or ABI Model 380B). In addition, antisense deoxyoligonucleotides have been shown to be effective in inhibiting gene transcription and viral replication (see e.g., Zamecnik et al., 1978 Proc. Natl. Acad. Sci. USA 75, 280-284; Zamecnik et al., 1986 Proc. Natl. Acad. Sci., 83, 4143-4146; Wickstrom et al., 1988 Proc. Natl. Acad. Sci. USA 85, 1028-1032; Crooke, 1993 FASEB J. 7, 533-539. Improved inhibition of expression of a gene by antisense oligonucleotides is possible if the antisense oligonucleotides contain modified nucleotides (see, e.g., Offensperger et. al., 1993 EMBO J. 12, 1257-1262 (in vivo inhibition of duck hepatitis B viral replication and gene expression by antisense phosphorothioate oligodeoxynucleotides); Rosenberg et al., PCT WO 93/01286 (synthesis of sulfurthioate oligonucleotides); Agrawal et al., 1988 Proc. Natl. Acad. Sci. USA 85, 7079-7083 (synthesis of antisense oligonucleoside phosphoramidates and phosphorothioates to inhibit replication of human immunodeficiency virus-1); Sarin et al., 1989 Proc. Natl. Acad. Sci. USA 85, 7448-7794 (synthesis of antisense methylphosphonate oligonucleotides); Shaw et al., 1991 Nucleic Acids Res 19, 747-750 (synthesis of 3′ exonuclease-resistant oligonucleotides containing 3′ terminal phosphoroamidate modifications).

The sequences of the 5′ flanking region of integrin protein gene can also be used in triple helix (triplex) gene therapy. Oligonucleotides complementary to gene promoter sequences on one of the strands of the DNA have been shown to bind promoter and regulatory sequences to form local triple nucleic acid helices which block transcription of the gene (see, e.g., 1989 Maher et al., Science 245, 725-730; Orson et al., 1991 Nucl. Acids Res. 19, 3435-3441; Postal et al., 1991 Proc. Natl. Acad. Sci. USA 88, 8227-8231; Cooney et al., 1988 Science 241, 456-459; Young et al., 1991 Proc. Natl. Acad. Sci. USA 88, 10023-10026; Duval-Valentin et al., 1992 Proc. Natl. Acad. Sci. USA 89, 504-508; 1992 Blume et al., Nucl. Acids Res. 20, 1777-1784; 1992 Grigoriev et al., J. Biol. Chem. 267, 3389-3395.

Both theoretical calculations and empirical findings have been reported which provide guidance for the design of oligonucleotides for use in oligonucleotide-directed triple helix formation to inhibit gene expression. For example, oligonucleotides should generally be greater than 14 nucleotides in length to ensure target sequence specificity (see, e.g., Maher et al., (1989); Grigoriev et al., (1992)). Also, many cells avidly take up oligonucleotides that are less than 50 nucleotides in length (see e.g., Orson et al., (1991); Holt et al., 1988 Mol. Cell. Biol. 8, 963-973; Wickstrom et al., 1988 Proc. Natl. Acad. Sci. USA 85, 1028-1032). To reduce susceptibility to intracellular degradation, for example by 3′ exonucleases, a free amine can be introduced to a 3′ terminal hydroxyl group of oligonucleotides without loss of sequence binding specificity (Orson et al., 1991). Furthermore, more stable triplexes are formed if any cytosines that may be present in the oligonucleotide are methylated, and also if an intercalating agent, such as an acridine derivative, is covalently attached to a 5′ terminal phosphate (e.g., via a pentamethylene bridge); again without loss of sequence specificity (Maher et al., (1989); Grigoriev et al., (1992).

Methods to produce or synthesize oligonucleotides are well known in the art. Such methods can range from standard enzymatic digestion followed by nucleotide fragment isolation (see e.g., Sambrook et al., Chapters 5, 6) to purely synthetic methods, for example, by the cyanoethyl phosphoramidite method using a Milligen or Beckman System 1Plus DNA synthesizer (see also, Ikuta et al., in An. Rev. Biochem. 1984 53, 323-356 (phosphotriester and phosphite-triester methods); Narang et al., in Methods Enzymol., 65, 610-620 (1980) (phosphotriester method). Accordingly, DNA sequences of the 5′ flanking region of the integrin protein gene described herein can be used to design and construct oligonucleotides including a DNA sequence consisting essentially of at least 10 to 15 consecutive nucleotides, with or without base modifications or intercalating agent derivatives, for use in forming triple helices specifically within the 5′ flanking region of a integrin protein gene in order to inhibit expression of the gene.

In some cases it may be advantageous to insert enhancers or multiple copies of the regulatory sequences into an expression system to facilitate screening of methods and reagents for manipulation of expression.

b. Carriers for Use with Compound Blocking Leukocyte Adherence and/or Function.

The compounds can be administered in any appropriate pharmaceutically acceptable carrier. Carriers for intravascular administration include saline, phosphate buffered saline or other comparable materials. Carriers for direct and/or topical administration include gels, foams, suspensions, microparticles, polymeric material and liposomes.

Carrier materials for direct administration include biodegradable materials, such as a synthetic polymer degrading by hydrolysis, for example, polyhydroxy acids like polylactic acid, polyglycolic acid and copolymers thereof, polyorthoesters, polyanhydrides, proteins such as gelatin and collagen, or carbohydrates or polysaccharides such as cellulose and derivatized celluloses, chitosan, alginate, or combinations thereof. Other materials include block copolymers of polyoxyethylene (Pluronics™, BASF) or the diacrylate block copolymers described by Hubbell, et al, in U.S. Pat. No. 5,567,435 issued on Oct. 22, 1996.

The use of biodegradable matrices eliminates the need for surgery to remove implanted materials. However, synthetic non-biodegradable matrices may also be used. Useful materials include ethylene vinyl acetate, polyvinyl alcohol, silicone, polyurethane, non-biodegradable polyesters, and tetrafluoroethylene meshes (Teflon®).

II. Methods for Inhibition of Leukocyte Adherence and Function

The method of treatment includes administering one or more of these compounds to a patient in need of treatment thereof. The compounds can be administered systemically or administered directly to the site of vascular injury prior to and/or at the time of injury and/or following the injury.

Systemic delivery can be performed by intraperitoneal administration, intravenous administration, intramuscular administration, intra-arterial administration, subcutaneous administration and oral administration.

Those of skill in the art can readily determine an effective concentration for treating a patient in need thereof typically based on extrapolation from animal data and from correlations established during clinical trials. Dosages will be dependent on the type of compound and route of administration. For example, in the case of monoclonal antibody suitable concentrations range from between 0.25 mg/Kg to 1 mg/Kg. Based on studies with other peptide fragments blocking receptor-mediated binding, the IC₅₀, the dose of peptide required to inhibit binding by 50%, ranges from about 50 μM to about 300 μM, depending on the peptides. These ranges are well within the effective concentrations for the in vivo administration of peptides, based on comparison with the RGD-containing peptides, described, for example, in U.S. Pat. No. 4,792,525 to Ruoslaghti, et al., used in vivo to alter cell attachment and phagocytosis.

Patients can be diagnosed for vascular injury using known methods, such as X-ray fluoroscopic examination of dye flowing through a particular region of a blood vessel or other visual techniques, the presence of symptoms such as pain, based on clinical judgment, or signs evidenced on physical examination. Alternatively, it can be assumed that injury will arise due to performance of procedures such as angioplasty, arterial bypass graft, peripheral bypass surgery, or organ transplantation and the patient treated based on the assumption that injury or disease will inevitably arise.

In general, this will result in a patient being treated systemically with the inhibitor of integrin mediated leukocyte adherence or function for between zero and 24 to 48 hours prior to surgery or vascular intervention, preferably about two hours, and for a period of time following surgery, typically until healing has occurred, which may be as long as six months following vascular intervention, although more typically will be for four to six weeks or until acute inflammation has subsided.

The following non-limiting example illustrates some of the various aspects of compositions and methods used to treat vascular smooth muscle cell hyperplasia and stenosis or restenosis of blood vessels.

### EXAMPLE 1

### Inhibition of Mac-1-dependent lignad binding in monocytes

Freshly isolated rabbit monocytes were incubated in the presence of M1/70 (available from the American Type Culture Collection TIB 128), which inhibits Mac-1 binding, and a control antibody, M5/14. M1/70 antibody concentration ranged from one to five μg/ml. Fibrinogen binding in the presence of the indicated antibodies was measured as a percentage of the control to determine the effectiveness of M1/70 in blocking Mac-1 dependent fibrinogen binding.

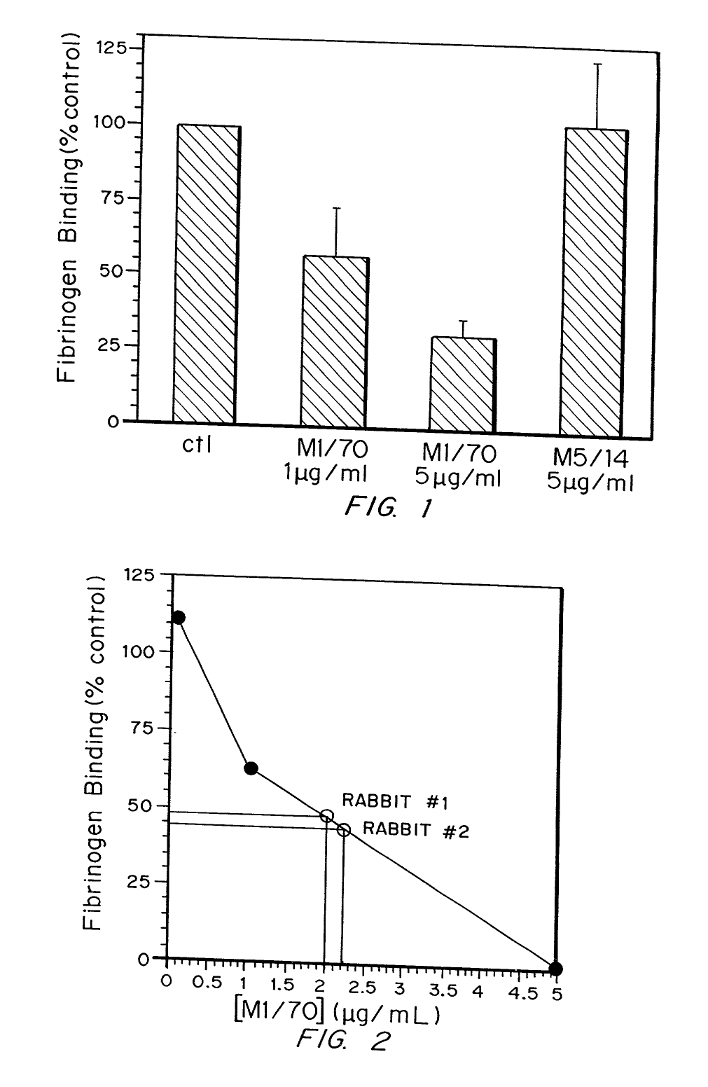

FIG. 1 is a graph of Mac-1-dependent ligand binding in rabbit monocytes showing % fibrinogen binding in the presence of M1/70 (1 and 5 μg/ml) or control mAb M5/14 (5 μg/ml), showing that the antibody was effective in a dose-dependent fashion to inhibit Mac-1 dependent fibrinogen binding.

FIG. 2 is a graph of Mac-1-dependent fibrinogen binding in cultured macrophages showing the degree of inhibition as a function of M 1/70 concentration. The serum level of M1/70 after intravenous bolus administration in two separate rabbits was estimated by extrapolating from the degree of inhibition of fibrinogen binding to Mac-1. The results demonstrate that intravenous bolus administration of M1/70 results in a serum level of approximately 2 to 2.5 μg/ml of M1/70 that is confirmed to inhibit Mac-1 function.

### EXAMPLE 2

### Inhibition of restenosis following vascular injury

M1/70 (1.0 mg/kg) (available from the American Type Culture Collection TIB 128) was administered intravenously to rabbits (1.0 mg/kg every other day) 2 hours prior to surgical intervention and for two weeks after arterial injury. Control infusions included normal saline or rat IgG. To mimic clinical syndromes, arterial injury consisted of balloon denudation of both iliac arteries via bilateral femoral arteriotomies followed by endovascular stent placement in one iliac artery. After two weeks, rabbits were sacrificed and arteries were pressure-perfusion fixed and examined histologically.

Neointimal hyperplasia after both superficial and deep injury was examined, and was significantly reduced with M1/70 treatment compared to both saline controls and IgG controls. After balloon angioplasty (superficial injury) neointimal area was reduced nearly 70% (FIG. 2A) relative to controls. The ratio of intimal:medial area, which is customarily used in balloon-injured experimental arteries to normalize for small normal variations in arterial size from one animal to another, was reduced over 75% relative to controls (FIG. 2B). After endovascular stent implantation (deep injury) neointimal area was reduced nearly 40% relative to controls (FIG. 2C). For reference, this is a profound inhibition of experimental restenosis by M1/70, equal to or greater than the inhibition achieved in this same animal model by “gold-standard” experimental antiproliferative agents such as heparin and others, discussed by Rogers, et al. Circulation 1993; 88:1215-1221, and further below.

This is the first demonstration of a treatment aimed directly at a single central cellular event being able to greatly impact vascular repair, inhibiting the neointimal growth which leads to restenosis. A specific molecular approach to inhibition of inflammatory cell recruitment as a means of modulating vascular cellular events has not been previously reported. The data indicates that blockade of Mac-1 favorably modulates vascular repair after superficial or deep vascular injury. This offers a marked advantage over existing pharmacologic and technical approaches.

Modifications and variations of the present invention will be obvious to those of ordinary skill in the art from the foregoing detailed description of the invention.

We claim:

1. A method of inhibiting or reducing stenosis or restenosis of a blood vessel following injury to vascular tissue in a region of the blood vessel of a patient in need of treatment thereof, comprising:
administering systemically or at the site of the injury a pharmaceutically acceptable composition comprising a compound which specifically inhibits or reduces leukocyte integrin-mediated adhesion or function in an amount effective to inhibit or reduce stenosis or dependent restenosis of a blood vessel following injury to vascular tissue.

2. The method of claim 1 wherein the leukocytes are monocytes or granulocytes.

3. The method of claim 1 wherein the injury arises from angioplasty, atherectomy, endovascular stenting, coronary artery bypass surgery, peripheral bypass surgery, or transplantation of cells, tissue or organs.

4. The method of claim 1 wherein the composition is in a form selected from the group consisting of solutions, gels, foams, suspensions, polymeric carriers, and liposomes.

5. The method of claim 1 wherein the integrin is selected from the group consisting of Mac-1, LFA-1, p150,95, and CD11d/CD18.

6. The method of claim 5 wherein the integrin is Mac-1.

7. The method of claim 6 wherein the ligand is selected from the group consisting of ICAM-1, fibrin(ogen), C3bi, and factor X.

8. The method of claim 1 wherein the compound is selected from the group consisting of antibodies and antibody fragments that are immunoreactive with integrins or their ligands and which block the interaction of the integrins or their ligands with vascular cells; molecules which inhibit expression of the integrins or their ligands, and peptides and peptidomimetics derived from the integrins or their ligands which block the interaction of the integrins or their ligands with vascular cells or tissues.

9. The method of claim 5 wherein the integrin is LFA-1 and the ligand is selected from the group consisting of ICAM-1, ICAM-2, ICAM-3.

10. The method of claim 6 wherein the compound is an antibody or antibody fragment immunoreactive with Mac-1.

11. The method of claim 1 wherein the compound is administered to a patient in need thereof prior to vascular intervention.

12. The method of claim 11 wherein the compound is administered to a the patient prior to and after vascular intervention, until healing has occurred.

13. A composition for inhibiting or reducing stenosis or restenosis of a blood vessel following injury to vascular tissue in a patient comprising an effective amount of a compound specifically inhibiting or reducing leukocyte adhesion or function mediated by an integrin selected from the group consisting of Mac-1, LFA-1, p150,95, and CD11d/CD18, to inhibit or reduce stenosis or restenosis of a blood vessel, wherein the compound is in a pharmaceutically acceptable carrier for administration to a vascular tissue.

14. The composition of claim 13 wherein the composition is in a form selected from the group consisting of solutions, gels, foams, suspensions, polymeric carriers, and liposomes.

15. The composition of claim 13 wherein the integrin is Mac-1.

16. The composition of claim 15 wherein the ligand is selected from the group consisting of ICAM-1, fibrin(ogen), C3bi, and factor X.

17. The composition of claim 13 wherein the compound is selected from the group consisting of antibodies and antibody fragments that are immunoreactive with integrins or their ligands and which block the interaction of the integrins or their ligands with vascular cells; molecules which inhibit expression of the integrins or their ligands, and peptides and peptidomimetics derived from the integrins or their ligands which block the interaction of the integrins or their ligands with vascular cell.


SEQUENCE LISTING (1 sequence)

SEQ ID NO: 1 - protein, 12 aa, Artificial Sequence
  Description of Artificial Sequence peptide
WPVFQKLRLDSV